(19) United States
(12) Patent Application Publication
(10) Pub. No.: US 2019/0350224 A1
(43) Pub. Date: Nov. 21, 2019

(54) METHOD FOR IMPROVING MUSCLE FORCE OR PHYSICAL FUNCTION

(71) Applicants: University of Washington, Seattle, WA (US); Astavita, Inc., Bellevue, WA (US)

(72) Inventors: Kevin Conley, Seattle, WA (US); Ziyang Liu, Seattle, WA (US); Yasuhiro Ogura, Bellevue, WA (US)

(73) Assignees: University of Washington, Seattle, WA (US); Astavita, Inc., Bellevue, WA (US)

(21) Appl. No.: 16/418,854

(22) Filed: May 21, 2019

Related U.S. Application Data
(60) Provisional application No. 62/674,329, filed on May 21, 2018.

(51) Int. Cl. A23K 20/142; A23L 33/175; A23L 33/105; A61K 9/00; A61K 38/06

(52) U.S. Cl. CPC A23K 20/142; A23L 33/175; A23L 2/52; A61K 9/0053; A61K 38/06; A23L 33/105

(57) ABSTRACT

The present invention relates to a method for improving a muscle force and a physical function, comprising combined use of an ingestion of a composition comprising one or more components selected from the group consisting of astaxanthin and its ester and a physical exercise.

### FIELD OF THE INVENTION

The present invention relates to methods for improving a muscle force and/or a physical function, and particularly to a method for improving and/or recovering a decreased muscle force or physical function.

### BACKGROUND OF THE INVENTION

In recent years, developed transportation systems and advanced technology of information and communication have caused lack of exercise in many people. Lack of exercise decreases a muscle force and muscle endurance, further decreasing a motor function as observed with sarcopenia. Further, when a muscle force decreases with increased age, a motor function also decreases potentially causing a fall or a fracture.

Physical exercises and training can be employed as a way of improving or recovering such a decrease in a muscle force. However, many people find it difficult to practice sufficient physical exercises or trainings due to a health reason, a temporal or a physical reason. Elderly people in particular often fail to practice sufficient trainings.

In response to this, supplements having an action to improve a muscle force and muscle endurance have been reported these days. For example, β-hydroxy-β-methylbutyrate (HMB), a metabolite of leucine, leucine, and arginine are reported to have an action to reinforce a muscle mass and a muscle force during training. Further, a composition containing royal jelly, milk whey protein, creatine and glutamine has been reported to have a muscle force improvement action.

As with other β-carotenoids, astaxanthin belongs to the carotenoid family and is a naturally occurring red pigment with abundant meal experiences, commonly found particularly in the ocean as in Crustacea such as shrimp and crab, fishes such as salmon and red snapper, algae such as the green alga Haematococcus, yeasts such as the red yeast Phaffia. In recent years, astaxanthin has been found to have an intense antioxidant action against reactive oxygen species (100 to 1,000 times more than vitamin E, about 40 times more than n-carotene) and has drawn attention as a material for health food products. Many other functional properties found in astaxanthin are reported such as an anti-inflammatory action, an anti-arteriosclerosis action, an anti-diabetes action, a retina protection action on photolesion, an anti-stress action and a sperm quality improvement action. Particularly, in regard with muscles, a method for treating exertional rhabdomyolysis in horses is known. Further, astaxanthin is also reported to have an action to ameliorate the muscular atrophy disorder.

### SUMMARY OF THE INVENTION

However, the metabolite of leucine, which is reported to increase a muscle mass, rather reduces exercise effects when used with an exercise, and thus the reinforcing actions of the leucine metabolite and other components are still dissatisfactory on a decreased muscle force and physical function.

Accordingly, it is an object of the present invention to provide a further outstanding method for improving a muscle force and a physical function.

Under the circumstances, the present inventors found that when an ingestion of astaxanthin and a physical exercise are used in combination, decreased muscle force and physical function are quite notably recovered or reinforced compared with the case of ingesting astaxanthin alone, whereby the present invention was accomplished.

More specifically, the present invention provides the following embodiments.

### Embodiment [1]

A method for improving a muscle force and/or a physical function comprising combined use of an ingestion of a composition comprising one or more components selected from the group consisting of astaxanthin and its ester and a physical exercise.

### Embodiment [2]

The method according to embodiment [1], wherein the composition is a composition comprising astaxanthin.

### Embodiment [3]

The method according to embodiment [1] or [2], wherein the ingestion is an oral ingestion.

### Embodiment [4]

The method according to any one of embodiments [1] to [3], wherein the physical exercise is a physical exercise which applies a load to a body muscle.

### Embodiment [5]

The method according to any one of embodiments [1] to [4], wherein the muscle force and/or the physical function is selected from the group consisting of a muscle mass, a muscle force during voluntary contraction, and a physical function.

### Embodiment [6]

The method for improving a muscle force and/or a physical function according to any one of embodiments [1] to [5], comprising recovering a decrease in a muscle force and a physical function.

### Embodiment [7]

The method for improving a muscle force and/or a physical function according to any one of embodiments [1] to [6], comprising applying the combined use of embodiment [1] to a patient with sarcopenia.

### Embodiment [8]

The method for improving a muscle force and/or a physical function according to any one of embodiments [1] to [6], comprising applying the combined use of embodiment [1] to an elderly individual.

### Embodiment [9]

The method for improving a muscle force and/or a physical function according to any one of embodiments [1] to [6], comprising applying the combined use of embodiment [1] to a patient with muscular atrophy.

### Embodiment [10]

The method for improving a muscle force and/or a physical function according to any one of embodiments [1] to [6], comprising applying the combined use of embodiments [1] to an elderly pet.

According to the present invention, the combined use of the ingestion of astaxanthin or its ester and a physical exercise notably improves a muscle force and/or a physical function, particularly by improving a muscle mass, a muscle force during voluntary contraction, a physical function and a physical function when compared with the case of only ingesting astaxanthin, additionally decreased muscle force and/or physical function is recovered, and further a decreased muscle mass is recovered. More specifically, in the present invention, a subject to which the above combined use is applied is a human who expects to improve a muscle force and/or a physical function. A healthy individual who aims health promotion may be a subject of the combined use. Subjects of the combined use who can expect higher effects include an elderly individual with a decreased muscle force or physical function, a human with a decreased muscle force or physical function who has been hospitalized for a long term due to an illness or an accident or who is discharged from a hospital and a human who is in a rehabilitation program to increase a muscle force or a physical function. A patient with sarcopenia or muscle atrophy can also expect improved effects when subjected to the combined use of the present invention. However, the improving effect on a muscle force and/or a physical function of the present invention may not be gained by a human who is unable to practice a physical exercise because the physical exercise may not be used in combination. Such a human who is unable to practice a physical exercise include a human with a severe muscle disease such as rhabdomyolysis or serious muscle atrophy disorder.

Further, as the subject of the combined use of the present invention, a small animal such as a dog and a cat, an anthropoid such as a monkey, a large animal such as a horse, in addition to the above human, can also expect the improving effect on a muscle force and/or a physical function or the recovering effect on a decreased muscle force and/or physical function. Thus, the invention is useful for treating elderly animals and pets.

### BRIEF DESCRIPTION OF THE DRAWINGS

FIG. 1 illustrates an apparatus for measuring muscle strength in ankle dorsiflexion.

FIG. 2 is a chart showing an example of a single maximum voluntary contraction at baseline and after three months of training in an Ax formulation treated human elderly subject.

FIG. 3 shows changes in endurance (training time) and mobility (walking distance) after 3 months of training in placebo (PL) and astaxanthin formulation (AX) treated elderly subjects. FIG. 3A shows the change in training time (min) in the recovery (REC) and interval (INT) exercise periods in the training session, and FIG. 3B shows the change in distance (meter) in the 6 min walk test.

FIG. 4 shows changes in muscle properties (maximum voluntary contraction (MVC), muscle cross-sectional area (CSA), and specific force (MVC/CSA)) after 3 month of training in Ax formulation and placebo treated elderly subjects.

FIG. 5 shows results in muscle performance test.

### DETAILED DESCRIPTION OF THE INVENTION

In the present invention, the “astaxanthin” refers to astaxanthin derived from a natural product or obtained by chemical synthesis. Examples of astaxanthin derived from natural products include astaxanthin obtained from a crust, an egg and an internal organ of Crustacea such as a shrimp, a krill or a crab, a skin or a roe of fishes, an alga such as a green alga Haematococcus, a yeast such as a red yeast Phaffia, a marine bacterium, a seed plant such as Amur adonis and Japanese Buttercup. Extracts from nature and chemically synthesized products are commercialized and readily available.

Astaxanthin can be obtained by, for example, culturing a red yeast Phaffia, a green alga Haematococcus or a marine bacterium in suitable medium in conformity with a known method. A green alga Haematococcus is the most preferable in light of ease in culturing and extraction, containing astaxanthin in the highest concentration and high productivity. The culturing method for obtaining a Haematococcus green algae with a high astaxanthin content is preferably a sealed culturing method being free from contamination and proliferation of heterologous microorganisms with little contamination of other impurities. For example, a culturing method which uses a semi-open dome-, cone- or cylindrical-shaped culture system and culture medium equipped with a gas discharge apparatus freely movable in the system (WO99/50384), a culturing method in which a light source is placed in a sealed culturing system which is irradiated from inside with light, and a culturing method which uses a tubular culture tank are suitable.

For the method of extraction and purification from the above culture or Crustacea, various methods are known such as an organic solvent extraction and a supercritical extraction. For example, with ester-type astaxanthin being an oil-soluble substance, an astaxanthin-containing component may be extracted from natural products containing astaxanthin using an oil-soluble organic solvent such as acetone, alcohol, ethyl acetate, benzene or chloroform. After extraction, the solvent may be removed in accordance with a routine method to obtain a mixed concentrate of astaxanthin monoester form and astaxanthin diester form. The obtained concentrate may further be purified as needed by a separation column or lipase decomposition.

Useful forms of astaxanthin, include the extract of astaxanthin obtained by the above method, for example as a powder or an aqueous solution containing the extract, or a dried product of red yeast Phaffia, green alga Haematococcus or marine bacterium and a crushed product thereof can be used.

Astaxanthin is 3,3′-dihydroxy-β,β-carotene-4,4′-dione, and it has optical isomers. Specifically, three optical isomers are known: (3R,3′R)-astaxanthin, (3R,3′S)-astaxanthin and (3S,3′S)-astaxanthin, any of which can be used in the present invention.

Mutagenicity is not observed with astaxanthin, which is thus known to be a very safe compound and has been widely used as a food additive (Jiro Takahashi et. al: Toxicity test on Haematococcus alga astaxanthin—Ames test, rat single administration toxicity test, rat 90 day-repeated oral administration toxicity test—, Journal of Clinical Therapeutics & Medicines, 20:867-881, 2004.).

In the description of the present invention below, astaxanthin, except the description in Examples, includes astaxanthin and/or esters thereof unless otherwise stated. Further, the ester of astaxanthin includes the monoester form and/or the diester form.

In the present invention, at least one of the free form, monoester form and diester form of astaxanthin can be used. In the diester form, two hydroxyl groups of the astaxanthin free form are replaced by ester-bonds, thus the diester form is physically more stable than the free form and the monoester form, and the diester form is hardly oxidatively decomposed. However, the diester form, when taken into the living body, is believed to be quickly hydrolyzed into the astaxanthin free form by in vivo enzymes to demonstrate effects.

Examples of the astaxanthin monoester form include esters in which one hydroxyl group of the astaxanthin free form is esterified by a lower or higher saturated fatty acid or a lower or higher unsaturated fatty acid. Examples of the lower or higher saturated fatty acid or the lower or higher unsaturated fatty acid specifically include acetic acid, lauric acid, myristic acid, pentadecanoic acid, palmitic acid, palmitoleic acid, heptadecanoic acid, elaidic acid, ricinoleic acid, petroselinic acid, vaccenic acid, eleostearic acid, punicic acid, licanic acid, parinaric acid, gadoleic acid, 5-eicosenoic acid, 5-docosenoic acid, cetoleic acid, erucic acid, 5,13-docosadienoic acid, selacholeic acid, decenoic acid, stering acid, dodecenoic acid, oleic acid, stearic acid, eicosapentaenoic acid, docosahexaenoic acid, linoleic acid, linolenic acid and arachidonic acid. Further, examples of the astaxanthin diester form include diesters obtained by esterification with the same or different fatty acid selected from the group consisting of the above fatty acids.

Further, examples of the astaxanthin monoester form include monoesters obtained by esterification with an amino acid such as glycine and alanine; monovalent or polyvalent carboxylic acid such as acetic acid and citric acid; inorganic acid such as phosphoric acid and sulfuric acid; sugar such as glycoside; sugar fatty acid such as glycero sugar fatty acid and sphingo sugar fatty acid; fatty acid such as glycero fatty acid; glycerophosphoric acid; or the like. If possible, salts of the above monoesters are also included.

Examples of the astaxanthin diester form include diesters in which two hydroxyl groups of the astaxanthin free form are esterified by the same or different acid selected from the group consisting of the above lower saturated fatty acid, higher saturated fatty acid, lower unsaturated fatty acid, higher unsaturated fatty acid, amino acid, monovalent or polyvalent carboxylic acid, inorganic acid, sugar, sugar fatty acid, fatty acid and glycerophosphoric acid. If possible, salts of the above diesters are also included.

For the diester form of the above glycerophosphoric acid, those in which the hydroxyl group of glycerophosphoric acid is ester-bonded to a saturated fatty acid or ester-bonded to a higher unsaturated fatty acid and an unsaturated fatty acid can also be used.

The composition used in the present invention may contain one or more components selected from the group consisting of astaxanthin and esters thereof and may contain other components. Examples of the other components which can be blended into the composition include a biologically active agent, a pharmaceutically acceptable carrier and a carrier acceptable in the food product field.

Examples of the biologically active agents include plant extracts containing flavonoids in the components such as SOD, mannitol, hydroquinone, bilirubin, cholesterol, tryptophan, histidine, quercetin, quercitrin, gallic acid, a gallic acid derivative, a gingko extract, vitamin A such as a gokahi extract, an Alnus firma fruit extract, a Lycii cortex extract, vitamin A acetate, vitamin A palmitate, a derivative thereof and a salt thereof, vitamin B, a derivative thereof and a salt thereof, vitamin C such as L-ascorbyl magnesium phosphate, disodium L-ascorbyl sulfate, vitamin C dipalmitate, a derivative thereof and a salt thereof, vitamin D, a derivative thereof and a salt thereof, vitamin E such as vitamin E acetate, a derivative thereof and a salt thereof, tocotrienol, a derivative thereof and a salt thereof, glutathione, a derivative thereof and a salt thereof, deoxyribonucleic acid and a salt thereof, an adenylate derivative such as adenosine triphosphate and adenosine monophosphate and a salt thereof, a ribonucleic acid and a salt thereof, a nucleic acid related substance such as guanine, xanthine, a derivative thereof and a salt thereof, an animal-derived extract such as a serum deproteinized extract, a spleen extract, a placenta extract, a cockscomb extract, royal jelly, a microorganism-derived extract such as a yeast extract, a lactic acid bacterial extract, a bifidus bacterial extract, a lingzhi mushroom extract, a plant-derived extract such as a carrot extract, a swertia extract, a rosemary extract, a cork tree bark extract, a garlic extract, hinokitiol and cepharanthine, α- or γ-linoleic acid, eicosapentaenoic acid and a derivative thereof, succinic acid, a derivative thereof and a salt thereof, estradiol, a derivative thereof and a salt thereof, an α-hydroxy acid such as lactic acid, glycolic acid, citric acid, malic acid and salicylic acid, a derivative thereof and a salt thereof, an inorganic nutrient such as zinc and magnesium and a salt of the inorganic substance and an organic acid such as zinc gluconate. Preferable examples of the above biologically active agents include vitamin C, a derivative thereof and a salt thereof, vitamin E, a derivative thereof and a salt thereof, tocotrienol and a derivative thereof, vitamin D and a salt thereof. The mixture of these biologically active substances and astaxanthin can further be formulated into an administration preparation such as a tablet and a capsule when combined, as needed, with a salt of the inorganic nutrient and a carrier acceptable in agents and the food product field as shown below.

The ratio of astaxanthin to the biologically active agent may be from 0.01 to 100 parts by weight of the biologically active agent to 1 part by weight of astaxanthin, preferably from 0.05 to 50 parts by weight of the biologically active agent to 1 part by weight of astaxanthin, most preferably from 0.1 to 20 parts by weight of the biologically active agent to 1 part by weight of astaxanthin.

For pharmaceutically acceptable carrier or carrier acceptable in the food field, examples of which include, more specifically, antioxidant compounds such as BHT and various carriers as shown below. These pharmaceutically acceptable carriers and carriers acceptable in the food product field are further described separately in the forms of an agent and food product.

Examples of preparation form used in the present invention include tablets, pills, granules, fine granules, powders, fine powders, capsules, microcapsules, nanocapsules, liquids, suspensions, emulsions and syrups, and an orally ingestible preparation forms are preferable.

For a component which can be blended, that is, for a carrier, various organic or inorganic carrier substances typically used in the pharmaceutical preparation field are used, and an excipient, a lubricant, a binder, a disintegrator, a solvent, a solubilizing agent, a suspending agent, a tonicity agent, a buffer, a preservative, an antioxidant, a colorant and a sweetener can be blended.

Examples of excipients are well known in the art including lactose, white sugar, glucose, D-mannitol, starch, corn starch, crystalline cellulose, magnesium aluminometasilicate, hydrotalcite, magaldrate, anhydrous dibasic calcium phosphate, light anhydrous silicic acid, gelatin, casein, various plant oils such as safflower oil and olive oil, bees wax and glycerin. Examples of the lubricant include magnesium stearate, calcium stearate, talc and colloidal silica. Examples of the binder include crystalline cellulose, white sugar, D-mannitol, dextrin, hydroxypropyl cellulose, hydroxypropyl methylcellulose and polyvinylpyrrolidone. Examples of the disintegrator include starch, crospovidone, carboxymethyl cellulose, carboxymethyl cellulose calcium, croscarmellose sodium, sodium carboxymethyl starch and agar. Examples of the solvent include water for injection, alcohol, propylene glycol, glycerin, macrogol, a sesame oil and a corn oil. Examples of the solubilizing agent include polyethylene glycol, propylene glycol, D-mannitol, benzyl benzoate, ethanol, tris aminomethane, cholesterol, triethanolamine, sodium carbonate and sodium citrate. Examples of the suspending agent include a surfactant such as stearyl triethanolamine, sodium lauryl sulfate, lauryl aminopropionate, lecithin, benzalkonium chloride, benzethonium chloride and glyceryl monostearate; a hydrophilic polymer such as polyvinyl alcohol, polyvinylpyrrolidone, carboxymethylcellulose sodium, methylcellulose, hydroxymethyl cellulose, hydroxyethyl cellulose and hydroxypropyl cellulose. Examples of the tonicity agent include sodium chloride, glycerin and D-mannitol. Examples of the buffer include a buffer solution such as phosphate, acetate, carbonate and citrate. Examples of the preservative include para-oxybenzonate, chlorobutanol, benzyl alcohol, phenethyl alcohol, dehydroacetic acid and sorbic acid. Examples of the antioxidant include sulfite, ascorbic acid, vitamin A, vitamin B, vitamin C, vitamin E or a vitamin derivative thereof, tocotrienol, cysteine, glutathione, gluthatione peroxidase, citric acid, phosphoric acid, polyphenol, a nucleic acid, a Chinese herbal medicine and a plant extract.

The above preparations can be produced in accordance with a routine method. Specifically, astaxanthin and the above biologically active agent as needed are formulated into oil or a micropowder using the above excipient and an additive and subsequently formulated into various dosage forms such as a soft capsule, a hard capsule or a tablet. Examples of the additive include sugar and sugar alcohol such as casein, gelatin, lactose, white sugar, glucose, D-mannitol, erythritol, xylitol, sorbitol and dextrin; and oil and fat such as bees wax and fatty acid triglyceride. Examples of the method for producing a micropowder include a spray-drying method, a fluidized bed granulation method, a kneading granulation method and a freeze-drying method.

Further, the preparation can be formulated into an intraoral quick disintegrating tablet by a routine method. For reducing suppressing bitterness and smell derived from the raw materials, an astaxanthin-containing micropowder or a final preparation may be coated, and the method for coating may follow a routine method. Further, for controlling the release of astaxanthin in the digestive tract, release control treatment can also be given.

Astaxanthin can be contained in an amount of from 0.01 to 99.9 wt %, preferably from 1 to 90 wt %, in the preparation form of the present invention. Astaxanthin or its ester blended into the preparation of the present invention may be orally administered typically in a daily dose of from 0.2 to 100 mg, preferably from 1.0 to 50 mg, to an adult in terms of astaxanthin free form. The daily dosage may be administered in a single dose, or in multiple doses, for example as two doses per day or three doses per day. The dosage may suitably be increased or decreased depending on age and body weight of a patient to be administered, symptoms and administration form.

The composition of the present invention can also be used as a food product (food and drink).

The composition can be used when added to a general food product as a carrier, which is a food and drink such as margarine, butter, butter sauce, cheese, whipped cream, shortening, lard, ice cream, yogurt, a dairy product, a sauced meat product, a processed seafood product, a fish product, a pickle, a noodle, a fried potato, a potato chip, a snack, a popcorn, a seasoned powder, a chewing gum, chocolate, pudding, jelly, a gummy candy, a candy, a drop, a caramel, bread, Castella (Japanese sponge cake), a cake, a donut, a biscuit, a cookie, a rice cracker and a cracker, macaroni, pasta, ramen, an udon noodle, a buckwheat noodle, salad oil, instant soup, dressing, an egg, mayonnaise and miso, or a carbonated or non-carbonated drink such as a fruit juice drink, a soft drink and a sport drink, a non-alcoholic drink such as tea, coffee and cocoa or an alcoholic drink such as liquor, medicinal liquor and fruit liquor.

Specifically, the food and drink of the present invention can be produced by blending astaxanthin or its ester with raw materials of the general food and drink and being processed in accordance with a routine method. The amount of astaxanthin or its ester blended varies depending on the form of food and drink and is not particularly limited but typically from 0.00001 to 10 wt %, preferably from 0.0001 to 5 wt %, and may be prepared in such a way that an amount required to demonstrate ameliorating actions is contained. The amount of astaxanthin or its ester used can be suitably selected by those skilled in the art depending on the type of food and drink, and astaxanthin or its ester is blended in such a way as to be ingested in an amount of about from 0.2 to 100 mg, preferably from 1.0 to 50 mg, daily per adult in terms of astaxanthin free form.

When the composition of the present invention is used as a nutritional complementary food product, a functional food product or a supplement, the form thereof may be the same as that of the above pharmaceutical preparations, and materials typically usable for food and drink can be blended. For example, milk protein, soy protein, egg albumen protein or decomposed products thereof such as egg white oligopeptide, soy hydrolyzate or a mixture of single amino acid can be blended. Further, processed products such as a natural liquid diet, a semi-digested nutritious diet and a nutritious diet, a drink, a capsule and an enteral nutrient, into which a sugar, fat, a trace element, a vitamin, an emulsifier or a flavor is blended, can also be blended. When the composition is provided in the form of a drink, a nutritional additive such as an amino acid, a vitamin, a mineral, a sweetener, a spice, a flavor and a pigment may be blended to enhance a nutritional balance and flavor when ingested.

The form of the composition of the present invention is preferably an orally ingestible form. A pharmaceutical composition for oral ingestion, or the form of supplement is particularly preferable.

The ingestion period of the composition in the present invention is not specifically limited. The ingestion may need to be continued until reinforcement of an intended muscle force and/or physical function or recovery of a decreased muscle force and/or physical function is achieved, and a specific period may be 1 week or more, 2 weeks or more, 1 month or more, or even 2 months or more, and may be suitably assessed and determined depending on age and condition of a subject such as a human who needs the ingestion.

The present invention relates to combined use of the ingestion of the above composition and a physical exercise. The combined use notably reinforces a muscle force and/or a physical function.

The physical exercise may only need to apply a certain level of load to a body muscle, which can be a normal load or above normal load, but may not need to be a real sport which can apply a high load to a body muscle. Examples of the physical exercise specifically include a training program in which a high-intensity and impulsive anaerobic exercise is intermittently repeated with incomplete recoveries in between, as represented by a high-intensity interval training. More specifically, an exercise load sufficient to achieve about from 75 to 80% of the maximum heart rate (HR) determined on an individual basis is continuously applied for about 20 seconds followed by a 10-sec break, and this may be repeated for about from 4 minutes to 30 minutes. For simply applying a load to achieve a HR from 75 to 80%, a human, for example, may intermittently repeat exercises such as walking, body stretching exercise, jogging, cycling, gymnastics or yoga or combinations thereof, further with high intense and low intense inserting there between as needed. Devices such as a treadmill and tools may also be used for these exercises. Further, when a high-intensity exercise may not be practiced due to a health reason, a moderate-intensity exercise may be practiced.

The high-intensity exercise described above refers to an exercise, which causes (1) harder and rougher breathing, (2) perspiration to start in 2, 3 minutes, and (3) difficulty in speaking with several or more words in a row, and of about from 75 to 80% of the maximum heart rate according to the criterion of heart rate.

Further, the moderate-intensity exercise refers to an exercise, which causes (1) faster breathing but not out of breath, (2) mild perspiration when continued for about 10 minutes, and (3) a conversation can be done but singing cannot, and of about from 50 to 75% of the maximum heart rate according to the criterion of heart rate. Examples of the typical moderate-intensity exercise include an 8,000-step walking in 20 minutes. In some embodiments, ball games such as tennis, badminton, table tennis and bowling may be practiced as long as the exercise content applies the load described above. Such a loading exercise may be practiced once a day and may be continued.

The above loading exercise is preferably continued basically in combination with the ingestion of the composition containing at least one or more components selected from the group consisting of astaxanthin and its ester until reinforcement of an intended muscle force and/or physical function or recovery of a decreased muscle force and/or physical function is achieved. Specifically, the period may be preferably 1 week or more, more preferably 2 weeks or more, further preferably 1 month or more, or may be even 2 months or more, and may be suitably assessed and determined depending on age and condition of a human who exercises.

In the present invention, the combined use of the ingestion of the composition and the physical exercise described above can notably improve a muscle force and/or a physical function. Examples of the improvement of a muscle force herein include, in the skeletal muscle, an increase in a muscle mass, improvement of a muscle force during voluntary contraction, and improvement of a muscle force per area, more specifically, a specific force. Further examples of the improvement of a physical function include improvement of endurance, improvement of mobility, and further specifically include improvement of whole body endurance and muscle endurance as represented by cardiopulmonary functions.

Subjects who can expect the improving effect on a muscle force and/or a physical function of the present invention include a healthy individual including an elderly individual who aims at health promotion, an elderly individual with a decreased muscle force or physical function, a human with a decreased muscle force or physical function who has been hospitalized for a long term due to illness, an injury or an accident or who is discharged from a hospital, a human who is in a rehabilitation program to increase a muscle force or a physical function, and a patient with sarcopenia or muscle atrophy can also expect more effects when subjected to the combined use of the present invention. The improving effect may not be gained with a human who is unable to practice a physical exercise, because the physical exercise may not be used in combination. Such a human who is unable to practice a physical exercise include a human with a severe muscle disease such as rhabdomyolysis or serious muscle atrophy disorder.

Further, as the subject of the combined use of the present invention, a small animal such as a dog and a cat, an anthropoid such as a monkey, a large animal such as a horse, in addition to the above human, can also expect the improving effect on a muscle force and/or a physical function or the recovering effect on a decreased muscle force and/or physical function.

### Examples

The present invention is described further in detail with reference to Examples below but the present invention is not limited thereto.

Astaxanthin subjected to a test is AstaREAL™ L10 oil (including 10% Natural Astaxanthin, marketed by AstaReal Co., Ltd.), which was extracted from a culture obtained by culturing Haematococcus alga, a species of the green algae, in a sealed culture system, concentrated and formulated into a gelatin capsule in accordance with a routine capsule production method with the following composition:

TABLE 1
Composition Contents (Astaxanthin Formulation)

| | |
|---|---|
| AstaREAL ™ L10 | 63.0 [mg/capsule] |
| Tocotrienol | 14.5 |
| Zinc gluconate | 25.0 |
| Safflower oil | 67.5 |
| Bees wax | 15.0 |
| Glycerin fatty acid ester | 15.0 |
| Total | 200.0 [mg/capsule] |

A capsule with the following composition was used as a placebo:

TABLE 2
Composition Contents (Placebo)

| | |
|---|---|
| Safflower oil | 168.0 [mg/capsule] |
| Bees wax | 16.0 |
| Glycerin fatty acid ester | 16.0 |
| Total | 200.0 [mg/capsule] |

### Example 1—Pilot Mouse Study

Twenty-nine month old male C57Bl/6NIA mice were treated with either 300 mg/(kg·day) astaxanthin (n=10, Astareal, Inc. Moses Lake, Wash., USA) or standard chow alone (n=9). The astaxanthin (Ax) dose was determined for mice by scaling [A simple practice guide for dose conversion between animals and human. J Basic Clin Pharm 7:27-31] the level found to be effective in rat studies [Int J Med Sci 8:126-138]. Exercise training occurred 3×/week at 20° incline on a treadmill at 10 m/min for 5 min at the start and reached 15 min for the final 4 weeks of training. In vivo muscle force of the gastrocnemius was measured as the maximum twitch and tetanic force during electric stimulations (200 Hz for 300 ms) at base line and 8 weeks in anesthetized mice as described [Aging Cell 12:763-771].

The quadriceps muscle was frozen at the end of training to determine the level of astaxanthin. The Institutional Animal Care and Use Committee of the University of Washington Animal approved this experimental protocol.

### Pilot Mouse Study Results:

An unpaired Student's t-test was used to evaluate treatment vs. placebo in the pilot mouse study. Significance was assigned at α=0.05 (P<0.05) without correcting for multiple comparisons consistent with a proof of concept study [BMJ 316:1236-1238]. Data are reported as means±SEM.

The level of astaxanthin in muscle after the 8-week exercise program was significantly elevated in the Ax (236.7±123.4 ng/g, n=4) vs. the placebo (9.2±9.2 ng/g, n=6) treatment group. Specific force (maximum twitch force/muscle mass) was significantly greater in Ax vs. placebo treated mice after training (P<0.05, Table 3).

TABLE 3
Mouse muscle force, size, and specific force (MVC/mass)
after 8 weeks of training with the Astaxanthin formulation
(AX) or placebo (PL) supplementation

| | | |
|---|---|---|
| PL | AX | P (PL vs. AX) |

Strength (max. force · N)

| | | | |
|---|---|---|---|
| Pre | 384 ± 21 | 364 ± 14 | >0.05 |
| Post | 366 ± 21 | 399 ± 26 | >0.05 |

Muscle Size (mass · g)

| | | | |
|---|---|---|---|
| Pre | 0.088 ± 0.01 | 0.081 ± 0.01 | >0.05 |
| Post | 0.090 ± 0.01 | 0.084 ± 0.01 | >0.03 |

Specific force (max force mass · N/g)

| | | | |
|---|---|---|---|
| Pre | 4.6 ± 0.3 | 4.3 ± 0.3 | >0.05 |
| Post | 4.0 ± 0.2 | 5.0 ± 0.2 | <0.01 |

Values are mean ± S.E.; Max. force: maximum twitch force elicited by electrical stimulation; CSA: cross sectional area; P: α level in a Student's t-test.

### Example 2—Human Study

Adults age 65-85 years old were recruited through public lectures, mailers, posted advertisements and referrals from prior studies. To be included in the study, subjects had to be: healthy and not under treatment for serious chronic conditions, ambulatory and able to perform activities of daily living without assistance, and able to speak and read English fluently. Exclusion criteria included:
1. Have significant disease(s) or condition(s) that put the subject at risk
2. Hospitalized within 3 months for major atherosclerotic events
3. Any metal implants in soft tissues
4. Implanted cardiac pacemaker or other win-MRI compatible implanted cardiac device
5. Have chronic. uncontrolled hypertension as judged by the Investigator
6. BMI (body mass index) of <18 or >32 kg/m2
7. Creatinine clearance <45 mL/min
8. Laboratory or ECG abnormalities
9. Clinically significant abnormalities on physical examination (as judged by the Investigator)
10. History or evidence of renal. hepatic, pulmonary (including chronic asthma). endocrine (e.g., diabetes. hypo- and hyperthyroidism. adrenal insufficiency). central nervous a neurologic disorders (MS, epilepsy, history of secures) or gastrointestinal (cirrhosis or viral hepatitis) system dysfunction.
11. Claustrophobia
12. Cancer, unless subject has documentation of completed curative treatment
13. History of serious mental illness as judged by the Investigator
14. Alcohol or drug abuse
15. Are currently or within the last 30 days enrolled in a clinical trial involving an investigational product or nonapproved use of a drug or device or concurrently enrolled in any other type of medical research judged not to be scientifically or medically compatible with this study
16. Donated a received blood or blood products within the past 30 days

Three hundred and sixty five subjects were phone screened and 58 subjects enrolled in the study and were randomly assigned to groups. Each subject had a physical examination, resting and exercise electrocardiogram and blood testing to ensure that they were healthy and free from orthopedic or neuromuscular problems. Sixteen subjects dropped out after randomization: 7 for medical reasons unrelated to the treatment, 5 for protocol non-adherence, and 4 for personal reasons. Forty-three subjects (n=19, placebo and n=23, astaxanthin formulation) aged 65-82 yr completed the study. The characteristics of the subjects that completed the study are shown in Table 4.

All participants gave written informed consent consistent with the Declaration of Helsinki in a project approved by the University of Washington and Western Institutional Review Boards.

TABLE 4
Subjects' characteristics

| | Placebo | AX |
|---|---|---|
| Age · Yr | 72.2 ± 1.2 | 69.1 ± 0.7 |
| F, n | 9 | 13 |
| M, n | 10 | 10 |
| Height · Cm | 66.6 ± 0.79 | 66.4 ± 0.86 |
| Weight · Kg | | |
| Pre | 71.1 ± 3.4 | 73.8 ± 2.8 |
| Post | 71.3 ± 3.4 | 72.4 ± 2.9 |
| BMI | | |
| Pre | 24.7 ± 0.71 | 26.3 ± 0.66 |
| Post | 25.4 ± 0.71 | 25.8 ± 0.66 |

Values measured are ± SEM

### Exercise Training:

The dietary formulation consisted of astaxanthin (12 mg), α-tocotrienol (10 mg) and zinc (6 mg) (prepared by Astamed, Bellevue, Wash.) and was ingested as 2 capsules per day. The 12 wk training program met 3× per week with a 10 min warm-up before and 5-10 min cool down period at the end of each session. Treadmill training involved walking at ˜1.3 msec with periods at a high treadmill incline of 9-12% grade (interval training) separated by periods of low incline walking at 5-7% grade (recovery). Table 5 contains the time and incline grade (%) used at baseline (Pre), at the end of training (Post) and the overall change with training. Training progressed in 3 steps: 1) familiarization with the treadmill protocol (weeks 1 & 2), which involved 8-10 interval sat the high incline at ˜1 min each and recovery for 2 min, 2) baseline interval training (weeks 3-7), which involved 1-1.5 min exercise in the 8-10 intervals to achieve 70-80% HR max with 2-3 min of recovery exercise between intervals and 3) ramping up (weeks 8-12), which involved 1-1.5-2 min exercise in the 10-12 intervals to achieve 80-85% HR max with 2-3 min of recovery exercise between intervals. All exercise training was overseen by an ACSM certified exercise physiologist at the Fred Hutchinson Cancer Research Center.

TABLE 5
Treadmill time (min) and incline grade (%) during training at baseline (Pre) at the end
(Post) and the change (Δ Post − Pre) with the study.

| | Pre | | Post | | ΔPost − Pre | |
|---|---|---|---|---|---|---|
| | Time | Grade | Time | Grade | Time | Grade |
| | (min) | (%) | (min) | (%) | (min) | (%) |
| PL-INT | 6.6 ± 0.9 | 8.8 ± 0.8 | 17.9 ± 0.8 | 9.9 ± 0.8 | 11.3 ± 1.1 | 1.1 ± 0.6 |
| PL-REC | 9.0 ± 0.0 | 4.8 ± 0.6 | 12.0 ± 0.9 | 7.0 ± 0.7 | 3.0 ± 0.9 | 2.2 ± 0.4 |
| AX-INC | 6.5 ± 0.5 | 9.5 ± 0.6 | 16.5 ± 1.0 | 10.8 ± 0.4 | 10.0 ± 0.9 | 1.4 ± 0.4 |
| AX-REC | 10.0 ± 0.7 | 5.3 ± 0.5 | 12.0 ± 0.8 | 6.4 ± 0.5 | 2.0 ± 1.0 | 1.0 ± 0.6 |

Values are means ± S.E.
Abbreviations are: PL—placebo.
AX—Astaxanthin formulation.
INT—interval.
REC—recovery.

### MR Imaging

Muscle size cross-sectional area was determined from MR images (Bruker 4.7-T magnet with Biospin console; Bruker Corporation, Billerica, Mass.) acquired as axial plane T1-weighted,2-D gradient-echo images collected with the following parameters: 500-ms repetition time, 2.5-ms echo time, 3-mm slice thickness, 1-mm inter-slice interval, 192×192 matrix, and number of excitations=2. Five slices of each right limb were analyzed with NIH Image software (Image J, version 1.50 e) using manual polarimetry [J Appl Physiol (1985) 90: 2070-2074] to determine the muscle CSA by two independent investigators who agreed in their measurements to within 2.5% on average.

### Single Muscle Test: Isometric Ankle Dorsiflexion

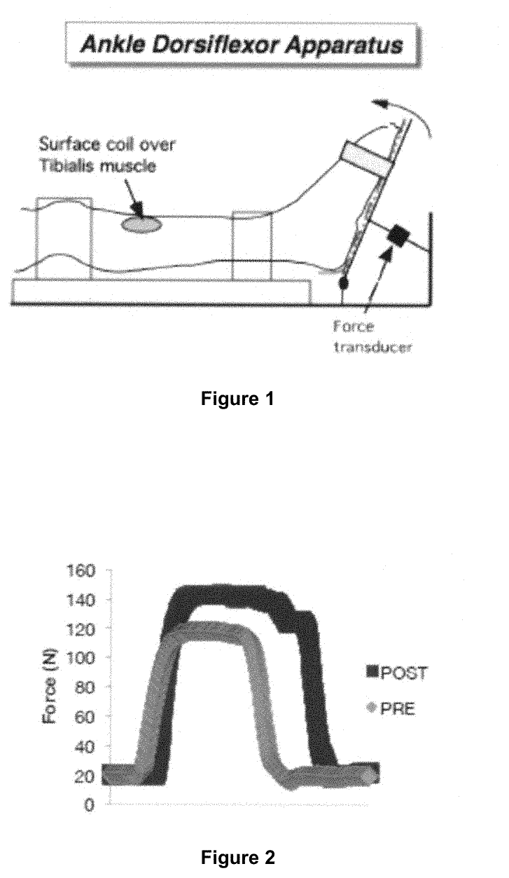

The TA muscle strength and contractile properties were determined on the right leg using a custom-built isometric exercise apparatus, as previously described [J Physiol 553:589-599] (FIG. 1). The subject performed a maximal voluntary contraction (MVC) in ankle dorsiflexion exercise for ˜5 seconds in 3 successive bouts by pulling on a strap that secured the foot to a force transducer platform. FIG. 2 shows a single maximum voluntary contraction at baseline and after three months of training in an Ax formulation treated human elderly subject.

### Statistical Analysis

A paired, 2-tailed t-test (pre- vs. post-training change) was used to evaluate treatment vs. placebo with exercise training in the human study. Significance was assigned at α=0.05 (P<0.05) without correcting for multiple comparisons consistent with a proof of concept study [BMJ 316:1236-1238]. Data are reported as means±SEM.

### Results

The subjects' physical characteristics are reported in Table 4, above.

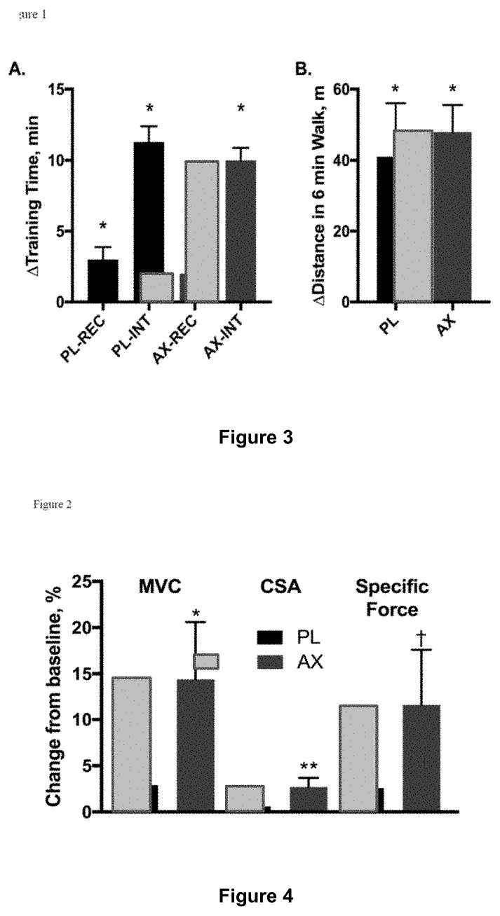

FIG. 3 shows that the time in the interval stage (high % grade incline walking) was the predominant change with training (See Table 5, above). The increased interval stage exercise time demonstrates that the subjects in both treatment groups could exercise longer (greater time) and at a higher intensity (higher % grade) after training. Walking distance in the 6 min walk also significantly improved by ˜8% in both groups with training (FIG. 3, Table 6).

TABLE 6
Walking distance at baseline (Pre), at the end (Post) and
the change (Δ Post − Pre) with the study in the placebo (PL) and
astaxanthin formulation (AX) fed groups.
Walking distance (Meter)

| | Pre | Post | Δ Post − Pre | P |
|---|---|---|---|---|
| P | 527 ± 17 | 568 ± 19 | 41 ± 15 | P < 0.001 |
| A | 530 ± 11 | 581 ± 11 | 48 ± 8 | P < 0.01 |

Values are mean ± SEM (paired t-test)

FIG. 4 shows the relative change in muscle strength and size in the two groups with training (Table 7 presents the absolute changes).

A significant change in human muscle strength as measured by MVC (Δ14.4±Δ6.2%, mean±SEM, P<0.02) is shown for the Ax treatment group alone. The TA muscle cross sectional area (CSA; Δ2.7±Δ1.0%) also increased only in the Ax treatment group (both image analyzers found CSA differences at P<0.01). The ratio of these measures provides the muscle specific force (MVC/CSA), which trended to a higher value (Δ11.6±Δ6.1%, P=0.053) in the Ax treatment group alone. No significant change in muscle properties was found in the placebo treatment group (MVC, Δ2.9%±Δ5.6%; CSA, Δ0.6%±Δ1.2%; MVC/CSA, Δ2.4±Δ5.7%; P>0.6 for all).

TABLE 7
Human TA muscle properties pre and post training and treatment
in the placebo (PL) and astaxanthin formulation (AX) fed groups

| | |
|---|---|
| PL | AX |

Strength (MVC, N)

| | | |
|---|---|---|
| Pre | 88.6 ± 5.9 | 83.2 ± 4.8 |
| Post | 87.4 ± 4.3 | 91.4 ± 4.0 |
| Δ Post − Pre | −1.2 ± 5.6 | 8.1 ± 3.8 |

Muscle Size (CSA · mm²)

| | | |
|---|---|---|
| Pre | 1030 ± 49 | 1064 ± 46 |
| Post | 1035 ± 49 | 1092 ± 48 |
| Δ Post − Pre | 5 ± 10 | 28 ± 10 |

Specific Force (MVC/CSA, N/mm^{a})

| | | |
|---|---|---|
| Pre | 0.09 ± 0.004 | 0.08 ± 0.005 |
| Post | 0.09 ± 0.005 | 0.09 ± 0.005 |
| Δ Post − Pre | −0 0002 ± 0.010 | 0.007 ± 0.004 |

Values are mean ± SEM; MVC maximal volunteer contraction force; CSA, cross sectional area

### Muscle Performance Testing:

Exercise tolerance was tested by measuring the sum of force generated by the TA muscle during repeated isometric contractions until exhaustion. The maximum voluntary contraction (MVC) was measured as the average of 3 maximum contractions separated by 5 sec and sustained for 3 sec each. The exercise level was set at 70% of the MVC and the exercise began at a frequency of 60 contractions per minute (cpm) for the first minute. This frequency was increased at a rate of 10 cpm with each minute until exhaustion. The force time integral (FTI) was measured as the sum of the force generated by these contractions. The total number of contractions was summed over the test.

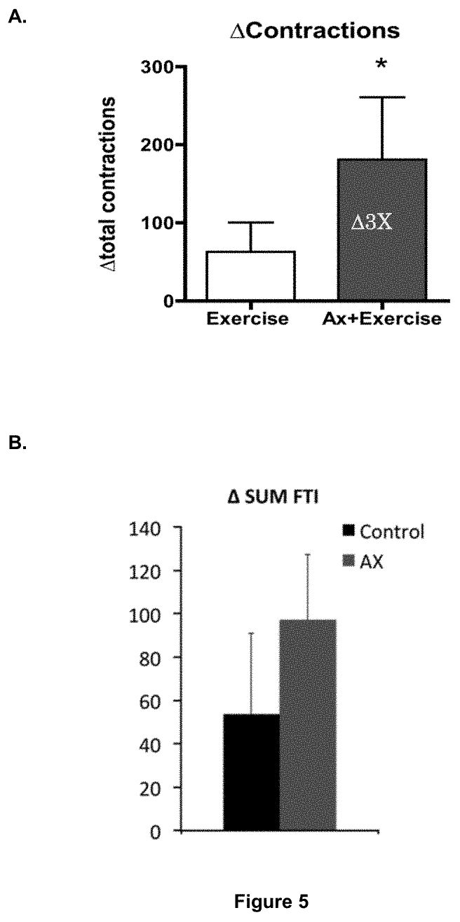

The performance test was not restricted by time. Instead, the subject exercised at a rate of contraction set by a metronome (starting at 60 contractions per minute) and the rate was raised each minute until the subject fatigued. For example, the test lasted ˜5 minutes if the subject fatigued during the 100 contractions per minute stage. If the subject completed the 60, 70, 80, and 90 cpm stages but stopped at 100 contractions per minute, then the sum of the contractions would be 60+70+80+90 or 300 contractions. Some subjects were able to exercise for a few seconds of a stage, so those contractions would be added to the total. The results are shown in the following Table 8 and the FIG. 5. FIG. 5A shows changes in muscle contractions between the PL and AX groups. FIG. 5B shows changes in force time integral (FTI) between the PL and AX groups.

TABLE 8
Muscle Performance (Endurance)
in a muscle performance test

| | |
|---|---|
| PL | AX |

Force Time Integral

| | | |
|---|---|---|
| Pre | 268.0 ± 33.0 | 397.3 ± 50.6 |
| Post | 370.3 ± 48.5 | 580.9 ± 116.2 |
| Δ Post-Pre | | |
| Number | 53.5 ± 37.5 | 102.3 ± 29.7 |
| Δ % | 26.2 ± 14.0 | 48.5 ± 14.2 |
| P value for paire | 0.15 | <0.01 |
| 2-trailed t-test | | |

Contractions (count)

| | | |
|---|---|---|
| Pre | 305.2 ± 30.8 | 371.2 + 30.4 |
| Post | 358.7 ± 42.1 | 435.7 ± 39.5 |
| Δ Post-Pre | | |
| Number | 73.4 ± 37.2 | 183.7 ± 76.6 |
| Δ % | 34.4 ± 12.3 | 50.0 ± 15.6 |
| P value for paire | 0.06 | 0.03 |
| 2-trailed t-test | | |

As apparently from the above results, functionally based exercise training combined with a formulation of natural anti-inflammatory and anti-oxidant compounds improved walking distance, exercise endurance and muscle strength and size, and muscle performance in elderly subjects more than exercise alone. These results suggest that the adaptive potential of elderly muscle for both strength and endurance improvements is apparent when natural products that promote adaptation are combined with an exercise approach having resistance and aerobic training components.

### Example 3—Preparation of Tablet

The following components were homogeneously mixed in the composition ratio (wt %) below to formulate a 180 mg tablet.

| | |
|---|---|
| Astaxanthin | 5% |
| Lactose | 75% |
| Heavy magnesium oxide | 20% |

### Example 4—Preparation of Intraoral Quick Disintegrating Tablet

The following components were homogeneously dry mixed in the composition ratio (wt %) below to formulate a 200 mg tablet by a routine compression.

| | |
|---|---|
| AstaREAL powder | 10.0% |
| F-MELT | 40.0% |
| Corn starch | 49.5% |
| Magnesium stearate | 0.5% |

AstaREAL powder [distributed by Fuji Chemical Industries Co., Ltd] is a powder containing 1% of astaxanthin in terms of free form, and F-MELT [distributed by Fuji Chemical Industries Co., Ltd] is an excipient containing sugar alcohol as the main component.

### Example 5—Preparation of Drink

The following components were blended to give a drink by adding water in accordance with a routine method.

| | | |
|---|---|---|
| Astaxanthin | 5 | g |
| Liquid sugar | 4 | kg |
| DL-sodium tartrate | 1 | g |
| Citric acid | 50 | g |
| Vitamin C | 50 | g |
| Vitamin E | 150 | g |
| Cyclodextrin | 25 | g |
| Potassium chloride | 5 | g |
| Magnesium sulfate | 2 | g |

What is claimed is:

1. A method for improving a muscle force and/or a physical function to a subject in need thereof, comprising combined use of an ingestion of a composition comprising one or more components selected from the group consisting of astaxanthin and its ester and a physical exercise.

2. The method according to claim 1, wherein the composition is a composition comprising astaxanthin.

3. The method according to claim 1, wherein the ingestion is an oral ingestion.

4. The method according to claim 1, wherein the physical exercise comprises applying a load to a body muscle.

5. The method according to claim 1, wherein the improving muscle force and/or the physical function is selected from the group consisting of a muscle mass, a muscle force during voluntary contraction, muscle endurance and whole body endurance.

6. The method for improving a muscle force and/or a physical function according to claim 1, wherein the subject is recovering from a decrease in a muscle force and a physical function.

7. The method for improving a muscle force and/or a physical function according to claim 1, wherein the subject is a patient with sarcopenia.

8. The method for improving a muscle force and/or a physical function according to claim 1, wherein the subject is an elderly individual.

9. The method for improving a muscle force and/or a physical function according to claim 1, wherein the subject is a patient with muscular atrophy.

10. The method for improving a muscle force and/or a physical function according to claim 1, wherein the subject is an elderly pet.
